SEQUENCE LISTING (12 sequences)

SEQ ID NO: 1 - protein, 12 aa, synthetic construct
  source 1..12: note = binding motif for FGFR3
VSPPLTLGQLLS

SEQ ID NO: 2 - protein, 13 aa, synthetic construct
  source 1..13: note = binding motif for Her2
FCGDGFYACYMDV

SEQ ID NO: 3 - protein, 14 aa, synthetic construct
  source 1..14: note = binding motif for IL-4Ra
KLAKLAKKLAKLAK

SEQ ID NO: 4 - protein, 12 aa, synthetic construct
  source 1..12: note = binding motif for EGFR
YHWYGYTPQNVI

SEQ ID NO: 5 - protein, 9 aa, synthetic construct
  source 1..9: note = linker sequence
GRAQGKAQG

SEQ ID NO: 6 - protein, 9 aa, synthetic construct
  source 1..9: note = linker sequence
GQAKGQARG

SEQ ID NO: 7 - protein, 9 aa, synthetic construct
  source 1..9: note = linker sequence
GKQAGRQAG

SEQ ID NO: 8 - protein, 9 aa, synthetic construct
  source 1..9: note = linker sequence
GQRAGQKAG

SEQ ID NO: 9 - protein, 87 aa, synthetic construct
  source 1..87: note = P1 conjugate sequence: E-I-H-F
YHWYGYTPQNVIGRAQGKAQGKLAKLAKKLAKLAKGQAKGQARGFCGDGFYACYMDVGKQAGRQAGVSPPLTLGQLLSGQRAGQKAG

SEQ ID NO: 10 - protein, 87 aa, synthetic construct
  source 1..87: note = P2 conjugate sequence: H-E-F-I
FCGDGFYACYMDVGRAQGKAQGYHWYGYTPQNVIGQAKGQARGVSPPLTLGQLLSGKQAGRQAGKLAKLAKKLAKLAKGQRAGQKAG

SEQ ID NO: 11 - protein, 10 aa, synthetic construct
  source 1..10: note = Human influenza hemagglutinin (HA) tag
YPYDVPDYAG

SEQ ID NO: 12 - protein, 29 aa, synthetic construct
  source 1..29: note = human influenza hemagglutinin (HA) tag
YPYDVPDYAGYPYDVPDYAGYPYDVPDYA

(19) United States
(12) Patent Application Publication
(10) Pub. No.: US 2025/0387497 A1
(43) Pub. Date: Dec. 25, 2025

(54) MULTIVALENT MULTISPECIFIC CONJUGATES AND RELATED COMPOSITIONS AND METHODS OF USE

(71) Applicant: Purdue Research Foundation, West Lafayette, IN (US)

(72) Inventor: Ruben Claudio Aguilar, West Lafayette, IN (US)

(21) Appl. No.: 19/128,281

(22) PCT Filed: Nov. 8, 2023
(86) PCT No.: PCT/US2023/079161

§ 371 Date: May 8, 2025

Related U.S. Application Data
(60) Provisional application No. 63/423,688, filed on Nov. 8, 2022.

(51) Int. Cl. A61K 47/64; A61K 47/69; A61P 35/00

(52) U.S. Cl. CPC A61K 47/642; A61K 47/6925; A61P 35/00

(57) ABSTRACT

A conjugate comprising (a) at least two or more binding motifs, each of which binds a different cell-surface molecule which is overexpressed or selectively expressed on a diseased (e.g., cancerous) cell, wherein adjacent binding motifs are separated from each other by a linker, which can be the same or different as a linker between other adjacent binding motifs, and (b) an active agent, which can be endocytosed by a cancerous cell to which the conjugate binds; a composition comprising the conjugate and a pharmaceutically acceptable carrier, a method of selectively targeting a cancerous cell in a subject for endocytosis of an anti-cancer agent; and a method of imaging a subject with cancer.

### PRIORITY

This patent application is related to and claims the priority benefit of U.S. Provisional Patent Application No. 63/423,688 filed Nov. 8, 2022. The content of the foregoing application is hereby incorporated by reference in its entirety into this disclosure.

### TECHNICAL FIELD

The present disclosure relates to a conjugate comprising binding motifs for cell-surface molecules overexpressed or selectively expressed on a target cell, linkers, and an active agent, compositions comprising same, and a method of using the conjugate or a composition comprising the conjugate for targeted delivery of an active agent and/or to treat a subject experiencing a disease state such as in cancer, genetic diseases and others.

### BACKGROUND

A number of therapeutic agents comprising targeting ligands linked to a drug contain one or more linkers placed between the targeting ligand and the therapeutically active drug. The targeting ligand can allow for specific binding to cells that express a cell surface molecule (e.g., a receptor, a transporter, a cell-cell communication protein, and others) for the ligand (e.g., cancer cells), resulting in delivery of the therapeutically active drug or imaging agent to the cells of interest. For example, specific binding of folate-drug conjugates to a folate receptor (FR) on cancer cells or inflammatory cells can allow for targeted delivery and specific therapeutic activity directed to the cancer cells or to sites of inflammation.

Most, if not all, meaning that the ligand-target complexes are stabilized by strong intermolecular forces, which ultimately leads to longer residence time at the binding site (higher “on” rate, lower “off” rate). Indeed, high-affinity binding ligands can be effective at binding cell surface molecules. However, these high-affinity ligands are typically not specific to a diseased cell (e.g., cancer cell). For example, FRs are expressed in a large number of cancers of epithelial origin, including breast, lung, kidney, and ovarian cancers. However, FR is also expressed in healthy tissues, such as at the brush-border membrane of the proximal tubule cells in a healthy kidney, which is accessible to drug conjugates in blood circulation.

Due to the bonding strength of high-affinity ligands with cell surface molecules and their presence on both diseased and healthy cells, conventional targeted drugs are not precise at discriminating between targeted and healthy (or otherwise off-target) cells, which can lead to off-target/secondary effect. For example, therapeutic application of many FR-targeted drugs is problematic due to high FR-mediated renal uptake. This can be especially problematic with imaging compounds that, for example, employ radioactivity.

Consequently, there is a need for targeting ligands and compounds with increased selectivity.

Two important unmet needs in cancer treatment are (i) the development of therapeutic agents affecting cancer cells while sparing normal cells (cancer selectivity) and (ii) the availability of effective strategies to treat variability in cell-surface markers between tumors within a single patient and between patients diagnosed with the same cancer (cancer variability). The failure of current strategies to address these aspects of cancer treatment contributes to poor outcomes, tumor resistance, and cancer recurrence.

In view of the above, it is an object of the present disclosure to provide materials and a method of treating targeted cells that seek to overcome the disadvantages of current strategies in addressing selectivity, in general, and cancer variability in particular. This and other objects and advantages, as well as inventive features, will be apparent from the detailed description provided herein.

### SUMMARY

Provided is a conjugate comprising (a) at least two or more binding motifs, each of which binds a different cell-surface marker which is overexpressed (or selectively expressed) on a targeted cell (e.g., a diseased cell such as, for example, a cancerous cell), wherein adjacent binding motifs are separated from each other by a linker, which can be the same or different than another linker between other adjacent binding motifs, and (b) an active agent, which can be endocytosed by a targeted cell to which the conjugate binds. Pharmaceutical salts of the conjugates are also provided. As noted above, when the conjugate comprises more than one linker, the linkers can be the same or different from each other.

In certain embodiments, the conjugate comprises a structure of Formula I:

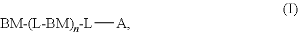

or is a pharmaceutically acceptable salt thereof, wherein each BM is one of the at least two binding motifs; each L is a linker; n is 1-5; and A is the active agent, n can be 5 or greater, n can be 1-3, n can be 2, n can be 5-10, n can be 3, n can be 4.

The targeted cell can be a diseased cell (e.g., a cancerous cell).

The conjugate can, and preferably does, comprise at least four binding motifs, each of which binds a different cell-surface molecule (e.g., a cell-surface receptor) which is overexpressed or selectively expressed on a targeted cell. Each of the at least four binding motifs can bind a different cell-surface molecule. The cell-surface molecule can be selected from the group consisting of a transporter, a receptor, and a cell-cell communication protein. In certain embodiments, each cell surface molecule is a cell surface receptor.

The conjugate can comprise at least four binding motifs, each of which binds a different cell-surface receptor which is overexpressed or selectively expressed on a targeted cell and is selected from the group consisting of fibroblast growth factor receptor 3 (FGFR3), Her2, interleukin-4 receptor alpha (IL-4Rα), and epidermal growth factor receptor (EGFR). In such embodiments, the targeted cell can be or comprise a cancerous cell.

The binding motif for FGFR3 can be VSPPLTLGQLLS (SEQ ID NO: 1) or a functional variant thereof (designated “F”), the binding motif for Her2 can be FCGDGFYACYMDV (SEQ ID NO: 2) or a functional variant thereof (designated “H”), the binding motif for IL-4Rα can be KLAKLAKKLAKLAK (SEQ ID NO: 3) or a functional variant thereof (designated “I”), and the binding motif for EGFR can be YHWYGYTPQNVI (SEQ ID NO: 4) or a functional variant thereof (designated “E”).

When the conjugate comprises more than one linker, each linker can be the same or different from each other. Each linker can be approximately 5 nm to 15 nm in length. Each linker can be approximately 7-10 nm in length. Each linker can be approximately 7 nm in length. In certain embodiments, each linker is approximately 7 nm in length and can be, and preferably is, flexible.

Each linker can have an amino acid sequence independently selected from GRAQGKAQG (SEQ ID NO: 5), GQAKGQARG (SEQ ID NO: 6), GKQAGRQAG (SEQ ID NO: 7), and GQRAGQKAG (SEQ ID NO: 8). Each linker can have an amino acid sequence independently selected from GRAQGKAQG (SEQ ID NO: 5), GQAKGQARG (SEQ ID NO: 6), GKQAGRQAG (SEQ ID NO: 7), and GQRAGQKAG (SEQ ID NO: 8), and each linker can be approximately 7 nm in length and flexible.

The active agent can be an anti-cancer therapeutic agent or an imaging agent. The active agent can be attached to a nanoparticle or encapsulated in a liposome, wherein the nanoparticle or liposome is attached to a linker of the conjugate. In certain embodiments, the active agent comprises an imaging agent. In certain embodiments, the active agent comprises a therapeutic agent.

The conjugate can comprise SEQ ID NO: 9 or a functional variant thereof.

The conjugate can comprise SEQ ID NO: 10 or a functional variant thereof.

The binding motif can be a low-affinity binding motif.

Also provided is a composition comprising a conjugate hereof or a pharmaceutically acceptable salt thereof, and a pharmaceutically acceptable carrier. The composition can comprise one conjugate hereof. The composition can comprise at least two different conjugates hereof, both of which comprise binding motifs that bind the same four cell-surface overexpressed or selectively expressed on a targeted cell but wherein the order of the binding motifs differs between the two conjugates. In certain embodiments, the composition comprises (i) a first conjugate comprising sequence YHWYGYTPQNVIGRAQGKAQGKLAKLAKKLAKLAKGQAKGQARGFCGDGFYACY MDVGKQAGRQAGVSPPLTLGQLLSGQRAGQKAG (SEQ ID NO: 9) or a functional variant thereof and (ii) a second conjugate comprising FCGDGFYACYMDVGRAQGKAQGYHWYGYTPQNVIGQAKGQARGVSPPLTLGQLLSG KQAGRQAGKLAKLAKKLAKLAKGQRAGQKAG (SEQ ID NO: 10) or a functional variant thereof.

Further provided is a method of selectively targeting a targeted cell (e.g., a cancerous or diseased cell) in a subject for endocytosis of a therapeutic agent (e.g., an anti-cancer agent). The method comprises administering to the subject an effective amount of a conjugate hereof or a pharmaceutically acceptable salt thereof, or a composition hereof, wherein the active agent of the conjugate or pharmaceutically acceptable salt thereof comprises an anti-cancer agent. In certain embodiments, when the cancerous cell endocytoses the therapeutic agent (e.g., anti-cancer agent), the endocytosis of the therapeutic agent (e.g., anti-cancer agent) treats the subject for cancer. The subject can have bladder cancer. The bladder cancer can be non-muscle invasive bladder cancer (NMIBC).

Still further provided is a method of imaging or diagnosing a subject with a disease state. The method comprises (a) administering to the subject an effective amount of a conjugate hereof or a pharmaceutically acceptable salt thereof, or a composition hereof, wherein the active agent of conjugate or pharmaceutically acceptable salt thereof comprises is an imaging agent; and (b) imaging the subject or having the subject imaged. The disease state can be cancer. The disease state can be bladder cancer. The disease state can be NMIBC. The disease state can be a genetic disease. The disease state can be fibrosis. The disease state can be an inflammatory disorder or disease state.

Imaging the subject can comprise performing radio-imaging, positron emission tomography (PET) imaging, single-photon emission computer tomography (SPECT) imaging, or magnetic resonance imaging. In certain embodiments, imaging the subject further comprises imaging an area of the subject affected by the disease state. The imaging agent can comprise a metal or isotope suitable for radio-imaging, PET imaging, SPECT imaging, or magnetic resonance imaging; or a fluorescent imaging agent, a photodynamic imaging agent, or an optical imaging agent.

In certain embodiments, use of the conjugates hereof (or pharmaceutically acceptable salts thereof), or a composition hereof in the preparation of a medicament for treating a disease state in a subject is provided. The disease state can be cancer. The disease state can be bladder cancer. The disease state can be NMIBC.

### DESCRIPTION OF THE DRAWINGS

The disclosed embodiments and other features, advantages, and aspects contained herein, and the matter of attaining them, will become apparent in light of the following detailed description of various exemplary embodiments of the present disclosure. Such detailed description will be better understood when taken in conjunction with the accompanying drawings.

FIG. 1 is a schematic diagram of the architecture of a bladder. GAG-glycosaminoglycan layer.

FIGS. 2A-2C demonstrate receptor micro-clustering. FIG. 2A shows multivalent agents, such as polyclonal antibodies (PAb), nanoparticles (NP), oligomerizing proteins (OPr), and multivalent peptides (MV) can induce receptor endocytosis by microclustering (μC) (grey, vertical ovals). FIG. 2B shows a multivalent and multispecific (MV-MS) conjugate can microcluster different receptors. Jack et. al., Int J Cancer 146(2): 449-460 (2020); Coon et al., Fibronectin attachment protein from bacillus Calmette-Guerin as targeting agent for bladder tumor cells, Int'l J Cancer, 131(3):591-600 (2012).

FIG. 2C shows examples of cargo internalized (darker concentration inside lighter endosomes) by μC. FAP: fibronectin attachment protein. PDL1: programmed death-ligand 1. EGF-PA: epidermal growth factor-protective antigen.

FIGS. 3A-3I illustrate the MV-MS targeting strategy.

FIG. 3A shows the average incidence of specific receptors overexpression in non-muscle invasive bladder cancer (NMIBC), wherein EGFR is epidermal growth factor receptor, ILR4a is interleukin-4 receptor alpha, FGFR3 is fibroblast growth factor receptor 3 and HER2 is human epidermal growth factor receptor 2. Joshi et al., Cancer Medicine 3(6): 1615-28 (2014); Hashizume et al., Oncotarget 9(75): 34066-34078 (2018); Rotterud et al., BJU Int'l 95 (9): 1344-1350 (2005).

FIG. 3B Left: Nanoparticles (NP) decorated with low density of conjugates do not favor μC to occur between but within MV-MS conjugates. FIG. 3B Right: Conjugates were produced by combining verified binding peptides against EGFR (E³⁰), interleukin-4 receptor alpha (IL-4Rα; I³¹), Her2 (H³²) and FGFR3 (F³³) with flexible linkers (each ˜7 nm in length) positioned in between and a human influenza hemagglutinin (HA) tag (e.g., YPYDVPDYAG (SEQ ID NO: 11) or YPYDVPDYAGYPYDVPDYAGYPYDVPDYA (SEQ ID NO: 12)) for immunodetection. Chen et al., Advanced Drug Delivery Review 65(10): 1357-69 (2013); Ching et al., Separation Science & Technology 24:7-8, 581-597 (1989).

FIG. 3C shows purified P1 and P2 peptides detected by Western blotting with anti-HA antibodies.

FIG. 3D shows that cells of human (T24), mouse (MB49^{LE}: only upregulating IL4R and FGFR3 receptors; WT: wild-type, which also expresses EGFR), and canine (isolated from spontaneous dog tumors and immortalized) origin bound and internalized P1/P2 peptides as revealed by anti-HA immunofluorescence. Arrows and arrowheads point to examples of internalized peptide in endosomal and late compartments, respectively. Inset in middle panel shows P1/P2 peptides within Rab5-positive endosomes.

FIG. 3E shows MB49 cells were synchronized, bound the peptides, and were induced to internalize the peptides. After five minutes, internalized peptide was localized to peripheral early endosomes (arrowheads) and after 45 minutes accumulated in late endosomes/lysosomes (arrows).

FIGS. 3F-3G show the amount of cell-associated peptide as a function of time as estimated by quantitative microscopy (FIG. 3F) and as a function of dose by quantitative Western blot (FIG. 3G) with anti-HA antibodies (upper panel) followed by band densitometry of triplicates (lower panel).

FIG. 3H shows ELISA-based testing of MV-MS binding to different extracellular domain (ECD) densities to emulate “normal” and “cancer” cells displaying one or two overexpressed (OE) receptors.

FIG. 3I shows GFP-Rab5^{Q79L}-expressing cells incubated with fluorescent nanoparticles decorated with nanomolar amounts of MV-MS (MVMS-NP). Internalized MVMS-NP was detected as signal inside endosome (arrows). Scale bars: 10 μm.

FIGS. 4A and 4B show images of immunostaining with an anti-HA antibody of normal (FIG. 4A) and MB49 wild type cancer (FIG. 4B) cells incubated with the highest concentration of P2 conjugates used.

FIG. 4C shows a graph of the quantification of cell-associated P2 (measured as total fluorescence intensity) per cell versus P2 dose.

While the present disclosure is susceptible to various modifications and alternative forms, exemplary embodiments thereof are shown by way of example in the drawings and are herein described in detail.

### SEQUENCE LISTINGS

The sequences herein (SEQ ID NOS: 1-12) are also provided in computer readable form encoded in a file filed herewith and incorporated herein by reference. The information recorded in computer readable form is identical to the written sequence listings provided herein, pursuant to 37 C.F.R. § 1.821(f).

### DETAILED DESCRIPTION

The present disclosure predicated, at least in part, on the discovery that multivalent conjugates that comprise two or more low-affinity binding motifs that simultaneously target two or more markers present on a targeted cell can result in a and significant increase in effective binding affinity (i.e., avidity) of the conjugate with respect to the targeted cell. Further, the individual low affinity binding motifs can be concatenated by intercalation of flexible linkers that provide an optimal separation between peptides. This linker-imposed separation can induce micro-clustering of the cell surface markers bound by the peptide units. Multivalent, multispecific (MV-MS) conjugates are provided that leverage these findings.

The MV-MS conjugates can be used for multiple applications where the selective targeting of cells is required and/or beneficial, such as in connection with the administration of treatment to and/or the targeted elimination of targeted cells. For example, the present conjugates can be used to selectively delivery therapeutics to specific kidney cells in connection with the treatment of genetic conditions or for the selectively delivery of a cytotoxic payload to cancer cells (e.g., glioma, medulloblastoma, bladder tumors, etc.). While particular examples are provided herein to facilitate understanding of the herein-described concepts, it will be appreciated that the conjugates, compositions and methods hereof can be used in connection with/for any application where selectively targeting a cell may be beneficial or desired.

In certain embodiments, a conjugate (or pharmaceutically acceptable salt thereof) is provided that comprises at least two or more binding motifs (BM), each of which binds a different cell-surface molecule (e.g., a transporter, receptor, cell-cell communication protein, etc.) which is overexpressed or selectively expressed on a targeted cell, and an active agent (A). Adjacent binding motifs can be separated from each other by a linker (L). Where, for example, the conjugate comprises multiple linkers (e.g., a first linker positioned between a first binding motif and a second binding motif, and a second linker positioned between the second binding motif and a third binding motif), each linker can be the same or different from each other.

In certain embodiments, the conjugate comprises the structure of Formula I:

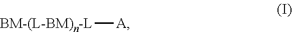

or a pharmaceutically acceptable salt thereof, wherein each BM is a binding motif, each L is a linker, n is an integer that is 1 or greater, and A is an active agent.

In certain embodiments, n is 1-5. In certain embodiments, n is 1-3. In certain embodiments, n is 1. In certain embodiments, n is 2. In certain embodiments, n is 3. In certain embodiments, n is 4. As noted above, each of the binding motifs can be different from each other such that each binds a different cell-surface receptor, and each linker can be the same or different from other linkers in the conjugate.

“Pharmaceutically acceptable salt” refers to a salt of a compound that retains the biological activity of the parent compound and which is not biologically or otherwise undesirable. Acid and/or base salts can be formed, for example, by reaction with amino and/or carboxyl groups. Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

The conjugates hereof, in application, can simultaneously target multiple upregulated or selectively expressed cell surface molecules (e.g., on a cancer cell or other targeted cell) and provide endocytosis control, enhanced selectivity for the targeted cells, and can even be highly effective against targeted cells with variable expression patterns (e.g., tumor cells) by eliciting self-adjusting affinity through, for example, coincidence detection of upregulated/selectively expressed molecules (i.e., with low- to normal-binding affinity, but resulting in high avidity for the targeted cells). Accordingly, the targeted cell can be a diseased cell. The targeted cell can be a cancerous cell. The targeted cell can be any cell with overexpress and/or selectively expressed cell-surface molecule(s) to which the binding motifs of the conjugate can be tailored.

### Low-Affinity Binding Motifs

As described above, the conjugate comprises at least two or more binding motifs. Each binding motif comprises a peptide or other moiety that corresponds with/binds a cell-surface molecule. As used herein, the terms “protein,” “polypeptide” and “peptide” refer to compounds comprising amino acids joined via peptide bonds and are used interchangeably. The binding motifs of the conjugate can be selected based on the type of cell that is targeted—i.e., to correspond with the upregulation or selective expression of two or more cell-surface targets of a particular cell. As used herein, “upregulation,” “overexpressed,” and their formatives (“up-regulated,” for example) are used interchangeably and refer to an increase in the level of a marker, such as a receptor, protein, or polypeptide as compared to a normal and/or healthy cell. The same consideration applies to “selectively expressed” cell surface molecules.

At least two of the binding motifs of the conjugate bind different cell-surface molecules. The conjugate can comprise at least three binding motifs, at least two of which bind different cell-surface receptors (i.e., in such case, at least two of the binding motifs are the same). In certain embodiments, the conjugate comprises at least three binding motifs, each of which binds a different cell-surface receptor which is overexpressed or selectively expressed on a targeted cell. The conjugate can comprise four or more binding motifs. In certain embodiments, the conjugate comprises at least four binding motifs, at least four of which binds a different cell-surface receptor which is overexpressed or selectively expressed on a targeted cell. In certain embodiments, the conjugate comprises at least four binding motifs, each of which binds a different cell-surface receptor which is overexpressed or selectively expressed on a targeted cell.

The binding motifs can be low-affinity peptides. As used herein, a “low-affinity binding motif” or “low-affinity peptide” means a peptide that, when complexed with a corresponding cell surface molecule or marker, results in only a weak interaction at the protein level. The complex resulting from an interaction between a low-affinity binding motif and a cell surface marker can have a dissociation constant (K_{D}) in the high micromolar range (e.g., ≥0.1 μM) or a fast kinetic off-rate (e.g., half-lives of less than about 1/second). For example, a low-affinity binding motif and cell surface marker complex can have a K_{D} from about 0.1 μM, or from about 1 μM, or from about 100 μM, to about 1000 μM, or to about 500 μM, or to about 250 μM, or to about 100 μM, or to about 10 μM. Thus, the affinity can be in the range from about 0.1 μM to about 1000 μM (or higher), or in the range from about 0.2 μM to about 900 μM, or in the range from about 0.3 μM to about 0.8 μM, or in the range from about 0.5 μM to about 700 μM, or in the range from about 10 μM to about 600 μM, or in the range from about 50 μM to about 500 μM, or in the range from about 100 μM to about 400 μM, or in the range from about 400 μM to about 500 μM, or in the range from about 1 μM to about 200 μM, or in the range from about 450 μM to about 480 μM, or in the range from about 10 μM to about 800 μM, or in the range from about 50 μM to about 100 μM, for example, as measured by Scatchard analysis, surface plasmon resonance technique (e.g., using BIACORE®) or equivalent techniques. The ranges set forth in this paragraph are inclusive of the stated end points and all 0.1 μM increments therein.

In certain embodiments, the binding motif can have a binding affinity for the cell-surface molecule of less than about 500 μM, such as 459 μM. In certain embodiments, the binding motif can have a binding affinity for the cell-surface molecule of less than about 0.5 μM, such as 0.3 μM. In certain embodiments, the binding motif can have a binding affinity for the cell-surface molecule of less than about 60 μM, such as 55.9 μM.

Cell surface molecules can comprise transporters, receptors, cell-cell communication proteins, and the like that are selectively expressed on the surface of a targeted cell. These molecules can be naturally endogenous to such cells or arise as a result of a mutation, such as for example in cancer cells. The cell surface molecules can be any molecules known to be upregulated or selectively expressed on a cell of interest as is known in the literature.

In certain embodiments, the cell-surface molecules comprise cell surface receptors overexpressed on the targeted cell. In certain embodiments, the cell surface receptors comprise fibroblast growth factor receptor 3 (FGFR3), Her2, interleukin-4 receptor alpha (IL-4Rα), and epidermal growth factor receptor (EGFR) and, optionally, the targeted cell is a cancerous cell. In certain embodiments, the cell surface molecule can comprise EGFRviii (e.g., a EGFR variant present in multiple gliomas, prostate, gastric, and other cancers). In certain embodiments, the cell surface molecule can comprise PDGFRα and/or PDGFRβ. In certain embodiments, the cell surface molecule can comprise Megalin (e.g., an endocytic receptor associated with kidney diseases).

Specific cell types can express particular combinations of upregulated or selectively expressed molecules on their cell surface. For example, certain cancer cells are known to overexpress FGFR3, Her2, IL-4Rα, and EGFR, each of which are receptors. When a particular cell type is known to upregulate or selectively express specific combinations of two or more cell surface molecules (e.g., 3 or 4 different molecules or markers), the low-affinity binding motifs of a conjugate can be strategically selected to correspond with that specific molecule combination to facilitate the targeted delivery and/or binding with such cell type.

As the binding motifs of the conjugate are low-affinity peptides, the presence of only one type of corresponding cell surface molecule, or a low density of the combination of cell surface molecules, present on a cell will result in weak or no binding. It is the synergistic combination of two or more different low-affinity binding motifs of the MV-MS conjugates and a high density or the selective expression of such molecules being present on the targeted cell that results in differential strong interaction. Accordingly, this causes MV-MS conjugates to display low affinity for normal or non-targeted cells (i.e., those not overexpressing the targeted combination of cell surface molecules).

Via the combination of binding motifs of the conjugates as described herein, a synergistic effect is produced; not only can the conjugates hereof bind a targeted cell (i.e., a cell expressing the combination of cell surface molecules that correlates with the combination of low-affinity binding motifs of the conjugate) with specificity, but when the low-affinity binding motifs of the conjugate simultaneously bind micro-clusters of molecules present on the targeted cell surface, the binding combination ultimately results in high binding avidity. This affinity switch is possible because certain cells (e.g., cancer cells) upregulate or selectively express two or more cell surface molecules that the conjugates hereof can simultaneously bind to yield high avidity. Targeting by coincidence detection as described herein is distinguishable from conventional multi-specific trends (e.g., bi-/tri-specific antibodies) because it can dynamically enhance the conjugate's effective affinity only when facing the targeted cells. In this manner, the MV-MS conjugates hereof can achieve the selective delivery of the active agent (A) to a targeted cell, thereby reducing off-target toxicity and other adverse effects associated with non-specific or less-specific delivery techniques.

As used herein, “specificity” refers to an interacting partner that recognizes only one another. For example, a binding motif binds “with specificity” to a receptor comprising a given receptor sequence if it binds to receptors comprising that portion of the amino acid sequence but does not bind to other receptors or proteins lacking that portion of the targeted sequence. A binding motif, or a conjugate comprising the binding motif, binds to its receptor (or a variant or mutein thereof) “with specificity” when it binds that receptor or a variant or mutein thereof with at least 2-fold greater, 3-fold greater, 4-fold greater, 5-fold greater, 6-fold greater, 7-fold greater, 8-fold greater, 9-fold greater, 10-fold greater, at least 15-fold greater, at least 20-fold greater, or at least 100-fold greater than its ability to bind any other receptor tested.

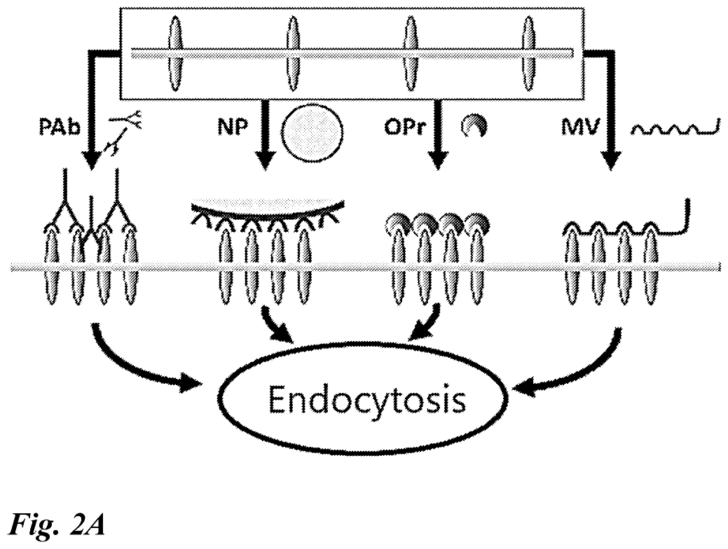

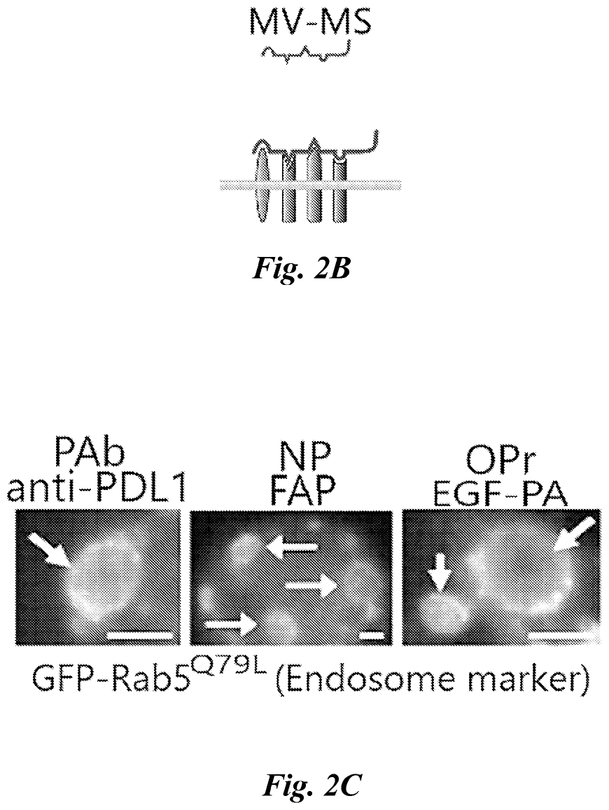

Additionally, the conjugates hereof can also induce endocytosis by and delivery of the active agent (e.g., a therapeutic agent) of the conjugate to targeted cells via receptor micro-clustering (uC) (FIG. 2A-2C). Coon et al. (2012), supra. Multivalent binding induces the formation of high local cargo densities, which can increase the number of endocytic sites and can accelerate initiation and maturation of endocytic vesicles. Liu et al., J Cell Biology 191(7): 1381-93 (2010); Pedersen et al., J Cell Biology 219(11): e202002160 (2020). Uptake by micro-clusters is insensitive to several types of receptor mutations affecting dimer formation and canonical endocytosis as well as other internalization-impairing scenarios (e.g., presence of Her2 for EGFR).

Further, the conjugates hereof can address challenges that result from tumor variability due to the multiple specificities of the conjugate binding motifs.

Specific examples of binding motifs can include, without limitation, a binding motif for FGFR3 being or comprising VSPPLTLGQLLS (SEQ ID NO: 1) or a functional variant thereof (designated “F”), a binding motif for Her2 being or comprising FCGDGFYACYMDV (SEQ ID NO: 2) or a functional variant thereof (designated “H”), a binding motif for IL-4Rα being or comprising KLAKLAKKLAKLAK (SEQ ID NO: 3) or a functional variant thereof (designated “I”), and a binding motif for EGFR being or comprising YHWYGYTPQNVI (SEQ ID NO: 4) or a functional variant thereof (designated “E”).

As used herein, a “functional variant” of an amino acid sequence or peptide is an amino acid sequence or peptide that can provide the same biological function as the reference sequence or peptide. In certain embodiments, the variants have less than 20, 11, 9, 8, 7, 6, 5, 4, 3, or less than 1 amino acid replacement as compared to the reference sequence or peptide.

Desirably, the sequences maintain about 90% to about 100% identity (e.g., 90% identity to about 100% identity, about 90% identity to 100% identity, or 90% identity to 100% identity), such as about 92.5% identity to about 97.5% identity (e.g., 92.5% identity to about 97.5% identity, about 92.5% identity to 97.5% identity, or 92.5% identity to 97.5% identity), or such as about 95% identity to about 96% identity (e.g., 95% identity to about 96% identity, about 95% identity to 96% identity, or 95% identity to 96% identity). The ranges set forth in this paragraph are inclusive of the stated end points and each 1% increment encompassed therein.

The term “identity” with respect to a reference to an amino acid or polypeptide sequence is defined as the percentage of amino acid or nucleic acid residues, respectively, in a candidate sequence that are identical with the residues in the reference sequence, after aligning the sequences and introducing gaps, if necessary, to achieve the maximum percent sequence identity and not considering any conservative substitutions as part of the sequence identity. Identity is measured by dividing the number of identical residues by the total number of residues and multiplying the product by 100 to achieve a percentage. Thus, two copies of exactly the same sequence have 100% identity, whereas two sequences that have amino acid deletions, insertions, or substitutions relative to one another have a lower degree of identity. Alignment for purposes of determining percent sequence identity can be achieved in various ways that are within the skill of the art, for instance, using publicly available computer software. For example, determination of percent identity or similarity between sequences can be done, for example, by using the GAP program (Genetics Computer Group, software; now available via Accelrys online), and alignments can be done using, for example, the ClustalW algorithm (VNTI software, InforMax Inc.). Further, a sequence database can be searched using the nucleic acid or amino acid sequence of interest. Algorithms for database searching are typically based on the BLAST software (Altschul et al., 1990), but those skilled in the art can determine appropriate parameters for aligning sequences, including any algorithms needed to achieve maximal alignment over the full length of the sequences being compared.

### Linkers

Adjacent binding motifs can be connected with a linker (L in Formula I). In certain embodiments, a binding motif and the active agent are connected with a linker. Where a conjugate comprises multiple linkers, the linkers can be the same or different (or any combination thereof).

Each linker can be any suitable linker. A linker can comprise atoms selected from C, N, O, S, Si, and P; C, N, O, S, and P; or C, N, O, and S. A linker can have a backbone that ranges in length, such that there can be as few as two atoms in the backbone of the linker to as many as 100 or more contiguous atoms in the backbone of the linker. The “backbone” of the linker is the shortest chain of contiguous atoms forming a covalently bonded connection between adjacent binding motifs and/or between a binding motif and an active agent. In some embodiments, a polyvalent linker has a branched backbone, with each branch serving as a section of backbone linker until reaching a terminus.

The linker can be non-releasable, i.e., non-labile. However, in some embodiments, it may be desirable for one or more linkers in a conjugate to be releasable, i.e., labile, such as, for example, photocleavable, acid-labile, base-labile, or enzyme-cleavable.

The length of each linker can be selected to optimize linker-imposed separation of molecules on the targeted cell surface, which in turn can induce micro-clustering of cell surface molecules bound by the binding motifs (e.g., when the conjugate is administered). The linker can have a chain length of at least about 5 nm. In certain embodiments, each linker is approximately 5 nm to 15 nm in length. In some embodiments, the linker is at least about 7 nm in length. In certain embodiments, each linker is approximately 7 nm in length and flexible. In certain embodiments, each linker is approximately 7-10 nm in length. In some embodiments, the linker is at least about 14 nm in length. In some embodiments, the linker is about 15 nm in length. In some embodiments, the linker is between about 7 nm and about 31 nm in length (such as, about 7 to 31, 7 to about 31, or 7 to 31), between about 7 nm and about 24 nm in length (such as, about 7 to 24, 7 to about 24, or 7 to 24), or between about 7 nm and about 20 nm in length (such as, about 7 to 20, 7 to about 20, or 7 to 20). In some embodiments, the linker is between about 14 nm and about 31 nm in length (such as, about 14 to 31, 14 to about 31, or 14 to 31), between about 14 nm and about 24 nm in length (such as, about 14 to 24, 14 to about 24, or 14 to 24), or between about 14 nm and about 20 nm in length (such as, about 14 to 20, 14 to about 20, or 14 to 20). In some embodiments, the linker has a chain length of at least 7 nm, at least 14 nm, at least 20 nm, at least 25 nm, at least 30 nm, or at least 40 nm; or from 5 nm to 15 nm, 5 nm to 10 nm, 7 nm to 10 nm, 5 nm to 20 nm, 10 nm to 40 nm, or 25 nm to 100 nm. In certain embodiments, the length of each linker is selected to facilitate micro-clustering of bound molecules on a cell surface to provide at or about 7-10 nm of separation therebetween (such as about 7 nm to about 10 nm, 7 nm to about 10 nm, about 7 nm to 10 nm, or 7 nm to 10 nm). The ranges specified in this paragraph are inclusive of the stated end points and all 1 nm increments encompassed within the stated ranges.

A linker can comprise at least one carbon-carbon bond and/or at least one amide bond. The linker can comprise one or more L- or D-configurations, natural or unnatural amino acids, or a combination of any of the foregoing.

In certain embodiments, a linker is a group comprising one or more covalently connected structural units.

In certain embodiments, a linker group is optionally substituted polyethylene glycol (PEG) having between 1 and about 100 ethylene glycol units. In certain embodiments, a linker is substituted with an aryl, phenyl, benzyl, alkyl, alkylene, or heterocycle group. In certain embodiments, a linker is asymmetric. In certain embodiments, a linker is symmetrical.

Alternatively, or in addition to chain length, in some embodiments, a linker can have suitable substituents that affect hydrophobicity or hydrophilicity. Thus, for example, a linker can have a hydrophobic side chain group, such as an alkyl, cycloalkyl, aryl, arylalkyl, or like group, each of which is optionally substituted. If a linker includes one or more amino acids, the linker can contain hydrophobic amino acid side chains, such as one or more amino acid side chains from Phe and Tyr, including substituted variants thereof, and analogs and derivatives of such side chains.

A linker can comprise a spacer (e.g., be conjugated with and/or include a spacer). The spacer can be any suitable spacer. A spacer of a linker can comprise hydrophilic, hydrophobic, amphipathic, non-peptidic, peptidic, and/or aromatic monomers. The length of a spacer can range from 1 to 30 (e.g., 1 to 30 carbon atoms, a PEG with 1-30 units, etc.). Examples of hydrophilic spacers include, but are not limited to, PEG polymers and derivatives thereof. Examples of hydrophobic spacers include, but are not limited to, pure or mixed branched hydrocarbons, fluorocarbons, alkane, alkene, and/or alkyne polymers. Examples of amphipathic spacers include, but are not limited to, pure or mixed phospholipids and/or derivatives thereof. Examples of peptidic spacers include, but are not limited to, pure and mixed single, branched, L- or D-configurations, essential, nonessential, natural, and unnatural amino acids and derivatives thereof. Examples of aromatic spacers include, but are not limited to, pure and mixed repeated quinoids.

In some embodiments a linker is formed via click chemistry/click chemistry-derived synthetic methods. Those of skill in the art understand that the terms “click chemistry” and “click chemistry-derived” generally refer to a class of small molecule reactions commonly used in conjugation, allowing the joining of substrates of choice with specific molecules. Click chemistry is not a single, specific reaction but describes a way of generating products that follow examples in nature, which also generate substances by joining small modular units. In many applications click reactions join a biomolecule and a reporter molecule. Click chemistry is not limited to biological conditions; the concept of a “click” reaction has been used in pharmacological and various biomimetic applications. However, they have been made notably useful in the detection, localization and qualification of biomolecules.

Click reactions can occur in one pot, typically are not disturbed by water, can generate minimal byproducts, and are “spring-loaded”-characterized by a high thermodynamic driving force that drives it quickly and irreversibly to high yield of a single reaction product, with high reaction specificity (in some cases, with both regio- and stereo-specificity). These qualities make click reactions suitable to the problem of isolating and targeting molecules in complex biological environments. In such environments, products accordingly need to be physiologically stable and any byproducts need to be non-toxic (e.g., for in vivo systems).

In certain embodiments, each linker has an amino acid sequence independently selected from GRAQGKAQG (SEQ ID NO: 5), GQAKGQARG (SEQ ID NO: 6), GKQAGRQAG (SEQ ID NO: 7), and GQRAGQKAG (SEQ ID NO: 8). In certain embodiments, each linker has an amino acid sequence independently selected from GRAQGKAQG (SEQ ID NO: 5), GQAKGQARG (SEQ ID NO: 6), GKQAGRQAG (SEQ ID NO: 7), and GQRAGQKAG (SEQ ID NO: 8), and is approximately 7 nm to 10 nm (e.g., 7 nm, 8 nm, 9 nm, or 10 nm) in length and flexible.

### Active Agent

The active agent can be attached to one end or the other of the chain of alternating binding motifs and linkers or to a side chain of an amino acid, such as an amino acid of which a linker is comprised. The active agent can be attached directly to the chain of alternating binding motifs and linkers, such as by covalent bonding or crosslinking. Alternatively, the active agent can be attached to a nanoparticle or encapsulated in a liposome, wherein the nanoparticle or liposome is attached to the chain of alternating binding motifs and linkers.

A “liposome” refers to a small, spherical vesicle composed of lipids, particularly vesicle-forming lipids that can spontaneously arrange into lipid bilayer structures in water with hydrophobic moieties in contact with the interior, hydrophobic region of the bilayer membrane, and head group moieties oriented toward the exterior, polar surface of the membrane. Vesicle-forming lipids typically have two hydrocarbon chains, particularly acyl chains, and a head group, either polar or nonpolar. Vesicle-forming lipids are either composed of naturally occurring lipids or synthetic lipids, including phospholipids, such as phosphatidylcholine, phosphatidylethanolamine, phosphatidic acid, phosphatidylinositol, and sphingomyelin, where the two hydrocarbon chains are typically between about 14 and 22 carbon atoms in length and have varying degrees of unsaturation. Such lipids and phospholipids can be obtained commercially or prepared according to published methods. Other suitable lipids include glycolipids and sterols, such as cholesterol and its various analogs. Cationic lipids, which typically have a hydrophilic moiety, such as a sterol, an acyl or a diacyl chain, and where the lipid has an overall net positive charge, also can be used in liposomes. The head group of the lipid typically carries the positive charge. The cationic vesicle-forming lipid also may be a neutral lipid or an amphipathic lipid derivatized with a cationic lipid. The liposomes can include a vesicle-forming lipid derivatized with a hydrophilic polymer to form a surface coating of hydrophilic polymer chains on the surfaces of the liposomes. The polymers can be homopolymers or block/random polymers. See, e.g., U.S. Pat. Nos. 5,395,619; 5,013,556; 5,631,018, and Int'l Pat. App. Pub. No. WO 98/07409, for preparation of vesicle-forming lipids derivatized with hydrophilic polymers.

The active agent can be or comprise a therapeutic (or prophylactic) agent, an imaging agent, a diagnostic agent, a compound that comprises a drug moiety or the like, and/or a pharmaceutically acceptable salt of such a compound or drug moiety.

In certain embodiments, the active agent is or comprises an imaging agent such as a radio-imaging agent, a radio-sensitizing agent, a radio-protecting agent, or a radiotherapeutic agent (e.g., covalently or non-covalently attached, directly or indirectly (e.g., via a nanoparticle) to the binding moieties via a linker).

Examples of imaging agents include, but are not limited to, a metal or isotope suitable for radio-imaging, positron emission tomography (PET) imaging, single-photon emission computer tomography (SPECT) imaging, or magnetic resonance imaging, a fluorescent imaging agent, a photodynamic imaging agent, or an optical imaging agent. The metal can be chelated with a chelating group selected from the group consisting of DOTA (1,4,7,10-tetraazacyclododecane-1,4,7,10-tetraacetic acid) or a derivative thereof, TETA (1,4,8,11-tetraazacyclotetradecane-1,4,8,11-tetraacetic acid) or a derivative thereof; SarAr (1-N-(4-Aminobenzyl)-3,6,10,13,16,19-hexaazabicyclo[6.6.6]-eicosane-1,8-diamine or a derivative thereof; NOTA (1,4,7-triazacyclononane-1,4,7-triacetic acid) or a derivative thereof; NETA (4-[2-(bis-carboxymethylamino)-ethyl]-7-carboxymethyl-[1,4,7]triazonan-1-yl) acetic acid or a derivative thereof; TRAP (1,4,7-triazacyclononane-1,4,7-tris[methyl (2-carboxyethyl)phosphinic acid) or a derivative thereof; HBED (N,N0-bis(2-hydroxybenzyl)-ethylenediamine-N,N0-diacetic acid) or a derivative thereof; 2,3-HOPO (3-hydroxypyridin-2-one) or a derivative thereof; PCTA (3,6,9,15-tetraazabicyclo[9.3.1]-pentadeca-1 (15), 11,13-triene-3,6,9,-triacetic acid) or a derivative thereof; DFO (desferrioxamine) or a derivative thereof; DTPA (diethylenetriaminepentaacetic acid) or a derivative thereof; OCTAPA (N,N0-bis(6-carboxy-2-pyridylmethyl)-ethylenediamine-N,N0-diacetic acid) or a derivative thereof; H₂-MACROPA (N,N′-bis[(6-carboxy-2-pyridipmethyl]-4,13-diaza-18-crown-6) or a derivative thereof; H2-DEDPA (1,2-[carboxy)-pyridin-2-yl]-methylamino]ethane or a derivative thereof; and an EC₂₀-head comprising β-1-diaminopropionic acid, aspartic acid, and cysteine. The metal chelating group can be bound to ¹¹C, ¹³C, ¹³N, ¹⁵O, ¹⁸F, ³²P, ⁴⁴Sc, ⁴⁷Sc, ⁵²Mn, ⁵⁵Co, ⁶⁰Co, ⁶⁴Cu, ⁶⁷Cu, ⁶⁷Ga, ⁶⁸Ga, ⁸⁶Y, ⁸⁹Sr, ⁸⁹Zr, ⁹⁰Y, ^{99m}Tc, ¹¹¹In, ^{114m}In, ^{117m}Sn, ¹²³I, ¹²⁴I, ¹²⁵I, ¹³¹I, ¹⁴⁹Tb, ¹⁵³Sm, ¹⁵²Tb, ¹⁵⁵Tb, ¹⁶¹Tb, ¹⁶⁹Er, ¹⁷⁷Lu, ¹⁸⁶Re, ¹⁸⁸Re, ²¹¹At, ²¹²Pb, ²¹²Bi, ²¹³Bi, ²²³Ra, ²²⁴Ra, ²²⁵Ab, ²²⁵Ac, or ²²⁷Th. The fluorescent imaging agent can be selected from the group consisting of carbocyanine, indocarbocyanine, oxacarbocyanine, thiacarbocyanine and merocyanine, polymethine, coumarine, rhodamine, xanthene, fluorescein, borondipyrromethane (BODIPY), CyS, CyS.S, Cy7, VivoTag-680, VivoTag-S680, VivoTag-S7S0, AlexaFluor660, AlexaFluor680, AlexaFluor700, AlexaFluor7S0, 10 AlexaFluor790, Dy677, Dy676, Dy682, Dy7S2, Dy780, DyLightS47, Dylight647, HiLyte Fluor 647, HiLyte Fluor 680, HiLyte Fluor 7S0. IRDye 800CW, IRDye 800RS, IRDye 700DX, ADS780WS, ADS830WS, ADS832WS and S0456.

The active agent can be or comprise a therapeutic agent. The term “therapeutic agent” is intended in its broadest meaning to include a compound, chemical substance, microorganism or any agent that is capable of producing an effect in a subject or on a living tissue or cell when administered thereto. Thus, the term includes both prophylactic and therapeutic agents, as well as diagnostic agents and any other category of agent capable of having a desired effect. Therapeutic agents include, but are not limited to, pharmaceutical drugs and vaccines, nucleic acid sequences (such as supercoiled, relaxed, and linear DNA and fragments thereof, antisense constructs, artificial chromosomes, RNA and fragments thereof, and any other nucleic-acid based therapeutic), cytokines, small molecule drugs, proteins, peptides and polypeptides, oligonucleotides, oligopeptides, fluorescent molecules (e.g., fluorophores) and other imaging agents, hormones, chemotherapy, and combinations of interleukins, lectins, and other stimulating agents.

In certain embodiments, the therapeutic agent is a chemotherapeutic agent. In certain embodiments, the therapeutic agent is a cytotoxic compound (e.g., a compound capable of disrupting cellular mechanisms that are important for cell survival and/or cell proliferation and/or causing apoptosis). In certain embodiments, the therapeutic agent comprises a therapeutic drug selected from the group consisting of an anti-cancer agent, a chemotherapeutic agent, a cytotoxic drug, an immunomodulator, an immunosuppressive agent, an immune activating agent, and/or an agonist.

Examples of therapeutic agents include, but are not limited to, erdafitinib, cisplatin, doxorubicin, monomethyl auristatin E, erdafitinib, mitomycin, pemigatinib, SN-38, thiotepa, valrubicin, the combination of cisplatin and gemcitabine, and MVAC (methotrexate, vinblastine sulfate, doxorubicin hydrochloride, and cisplatin).

The immunomodulator can be any suitable immunotherapeutic drug. Examples of suitable immunotherapeutic drugs include, but are not limited to, a transforming growth factor beta (TGF-β) inhibitor, such as R268712.

The anti-cancer agent can be any suitable anti-cancer drug. Examples of suitable anti-cancer drugs include, but are not limited to, a kinase inhibitor, such as dasatinib.

The chemotherapeutic agent can be any suitable chemotherapeutic drug. Examples of suitable chemotherapeutic drugs include, but are not limited to, an anthracycline, such as doxorubicin, taxane, such as docetaxel, cyclophosphamide, such as Cytoxan, or 5-fluoro-uracil.

The conjugate can comprise SEQ ID NO: 9 or a functional variant thereof. The conjugate can comprise SEQ ID NO: 10 or a functional variant thereof. Such sequences can tolerate minor modifications as discussed above.

When targeting cells other than cancerous cells (e.g., diseased or genetically modified cells), the active agent can be any type of therapeutic agent effective against the specific condition or cells targeted.

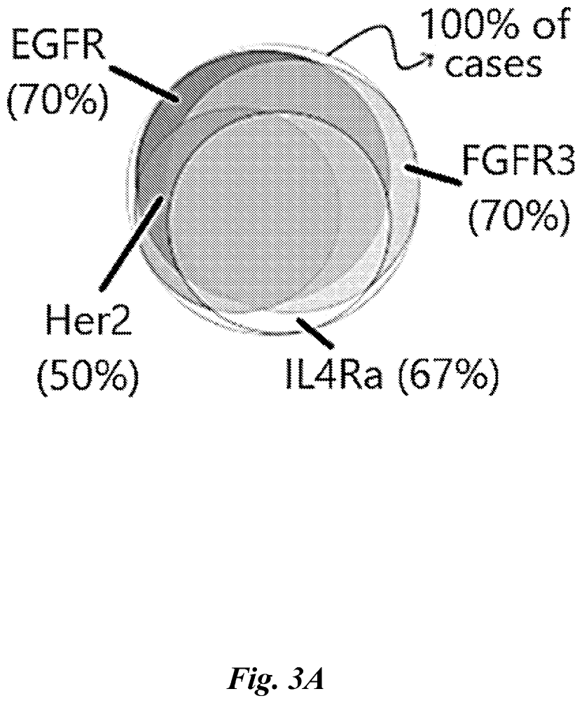

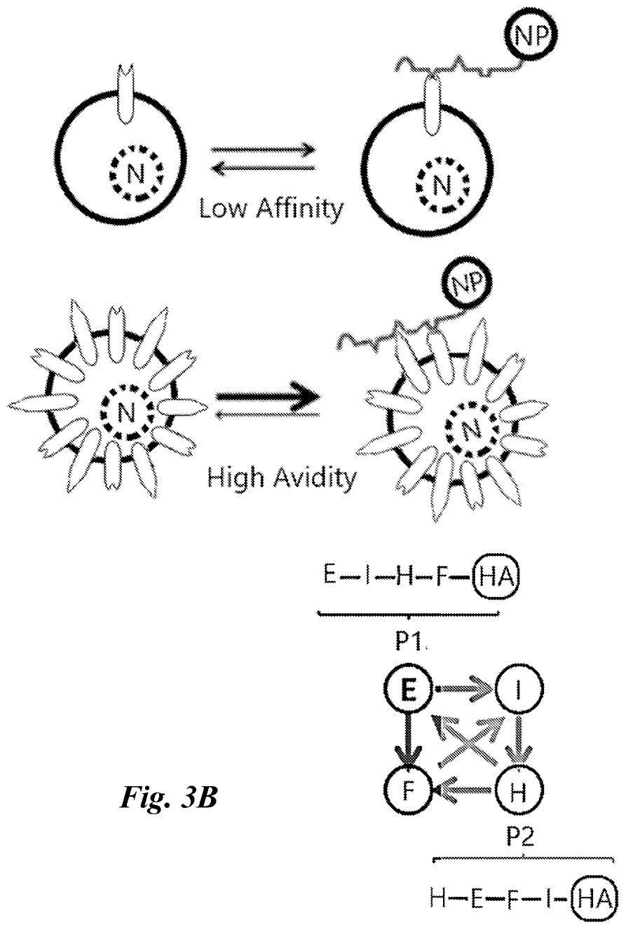

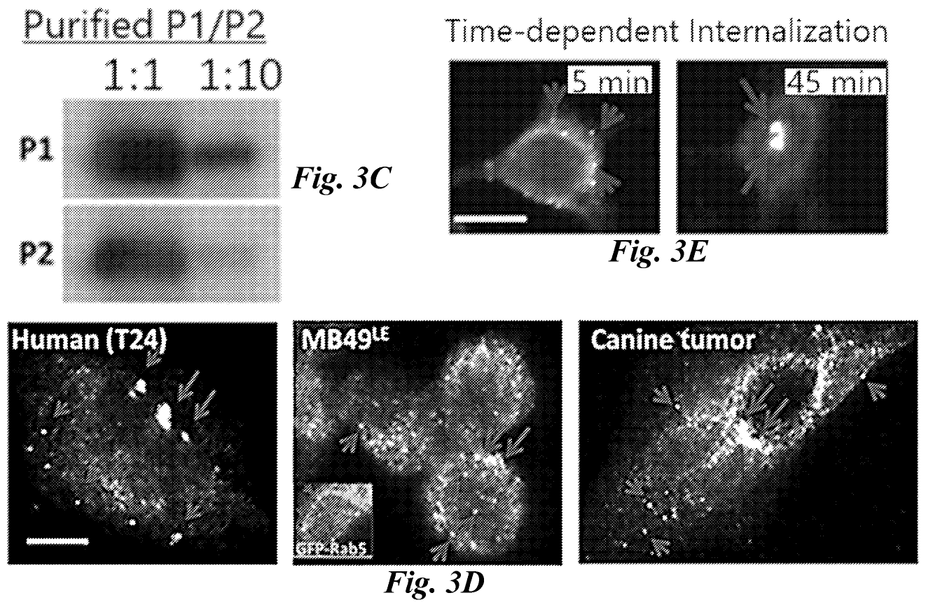

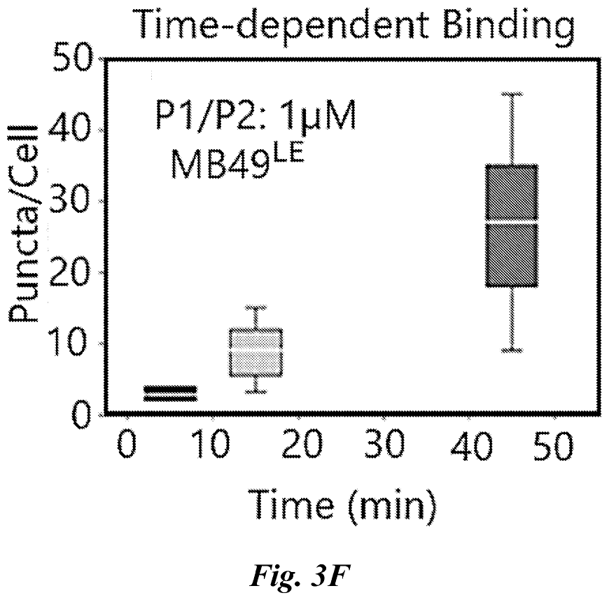

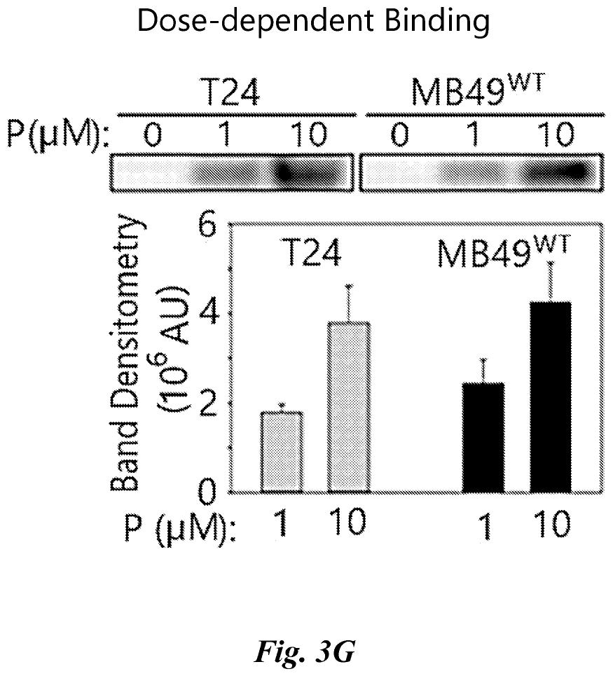

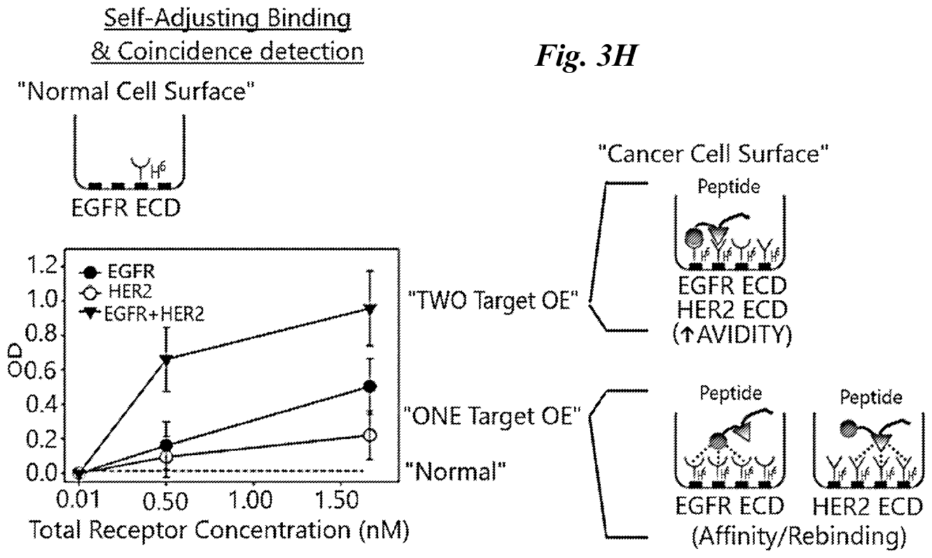

The conjugates can be synthesized in accordance with methods known in the art. Solid-phase synthesis, solution-phase synthesis, or a combination of both can be used. Peptide chains can be assembled on solid phase, and cyclization or other modifications can be performed on resin or in solution (see, e.g., Chan and White, Fmoc Solid Phase Peptide Synthesis, A Practical Approach, Oxford University Press (2000), and references cited therein). The conjugates are easily purified in high yields. See, e.g., FIG. 3C.

### Compositions

In view of the above, also provided is a composition comprising an above-described conjugate and a pharmaceutically acceptable carrier. “Pharmaceutically acceptable carrier” includes any of the standard pharmaceutical carriers, such as, but not limited to, a buffering agent, a preserving agent, an anesthetic agent, a solubilizing agent, an isotonic agent, a wetting agent, and a stabilizer. The term also encompasses any of the agents approved by a regulatory agency, such as the U.S. Food and Drug Administration, or listed in the U.S. Pharmacopeia for use in animals (e.g., mammals, such as humans). The carrier can be a phosphate-buffered saline solution, water, or an emulsion such as an oil/water or water/oil emulsion.

The composition can comprise a pharmaceutically acceptable salt, hydrate, or solvate of a conjugate.

The composition can comprise at least two different conjugates, both of which comprise binding motifs that bind the same four cell-surface molecules overexpressed or selectively expressed on a targeted cell (e.g., a cancerous cell) but wherein the order of the binding motifs differs between the two conjugates. The composition can comprise (i) a first conjugate comprising SEQ ID NO: 9 or a functional variant thereof and (ii) a second conjugate comprising SEQ ID NO: 10 or a functional variant thereof. Such sequences and functional variants can tolerate minor modifications as discussed above.

### Uses and Methods

Further provided is a method of selectively targeting a cell (e.g., a diseased cell, a cancer cell, or another type of cell) in a subject for endocytosis of an active agent. The method can comprise administering to the subject an effective amount of any of the conjugates or compositions described herein. As used herein, a “subject” means any warm-blooded, vertebrate, domesticated animal (e.g., livestock, horses, cats, and dogs) and humans.

An “effective” amount is an amount of an active agent that provides a desired effect, such as an anti-cancer effect or the ability to image cancerous cells in a subject. The amount that is “effective” will vary from subject to subject, depending on the age and general condition of the individual, mode of administration, and the like. Thus, it is not always possible to specify an exact “effective amount.” An appropriate “effective amount,” however, can be determined by one of ordinary skill in the art using routine experimentation.

In certain embodiments, the method comprises selectively targeting a cancerous cell in a subject for endocytosis of an active agent comprising administering an effective amount of (a) a conjugate or pharmaceutically acceptable salt hereof, or (b) a composition hereof, wherein the active agent of the conjugate or pharmaceutically acceptable salt comprises an anti-cancer agent. The cancerous cell can endocytose the active agent (e.g., the anti-cancer agent), wherein the endocytosis of the active agent treats the subject for a disease state, such as cancer. The subject can have bladder cancer (e.g., transitional cell carcinoma, squamous cell carcinoma, adenocarcinoma, and combinations thereof). In certain embodiments, the bladder cancer is NMIBC. When the cancerous cell endocytoses an anti-cancer agent, the endocytosis of the anti-cancer agent can treat the subject for cancer.

It will be appreciated that the disease state can comprise any disease state where a targeted cell overexpresses or selectively expresses two or more cell-surface molecules that can be targeted by the binding motifs of the conjugates hereof.

In certain embodiments, the method comprises selectively targeting a diseased cell in a subject for endocytosis of a therapeutic agent comprising administering an effective amount of (a) a conjugate hereof, or (b) a composition hereof, wherein the active agent of the conjugate comprises a therapeutic agent. The diseased cell can endocytose the therapeutic agent, wherein the endocytosis of the therapeutic agent treats the subject for the disease.

The composition can be administered by any suitable route as known in the art. For administration to a subject with bladder cancer, the composition can be administered directly to the lumen of the bladder, such as by injection or catheterization. In certain embodiments where the subject is experiencing a genetic condition related to specific kidney cells, the composition can be administered (and formulated to be administered) directly to the kidney and/or via injection into the blood stream.

“Treat,” “treating,” “treated,” and “treatment” are used to refer to the prevention, alleviation or elimination of symptoms and signs associated with a specific disease, disorder or condition (e.g., cancer). “Treating” cancer, for example, can include maintaining, reducing, or eliminating detectable cancer from a subject, such as a patient, and/or inhibiting or preventing the metastasis thereof.

Still further provided is a method of imaging or diagnosing a subject with a disease state. In certain embodiments, the subject has cancer. The method can comprise (a) administering to the subject an effective amount of an above-described conjugate, a pharmaceutical salt thereof, or a composition described herein, wherein the active agent of the conjugate or pharmaceutically acceptable salt thereof comprises an imaging agent; and (b) imaging the subject, or having the subject imaged. In certain embodiments, the disease state is cancer.

Imaging the subject can comprise performing radio-imaging, PET imaging, SPECT imaging, magnetic resonance imaging, or using any other imaging technique known in the art. Imaging the subject can further comprise imaging an area of the subject affected by the disease state (e.g., a tumor location, kidneys where the subject is experiencing a genetic condition that affects the kidneys, etc.). In certain embodiments, the imaging agent comprises a metal or isotope suitable for radio-imaging, PET imaging, SPECT imaging, or magnetic resonance imaging; or a fluorescent imaging agent, a photodynamic imaging agent, or an optical imaging agent.

It will be appreciated that the selective targeting of the conjugates and compositions hereof can be used to identify the location of the targeted cells within a subject using imaging techniques. Additionally, or alternatively, the conjugates and compositions hereof can be used to diagnose a disease state. For example, where a conjugate hereof is administered to a subject, wherein such conjugate targets a specific targeted cell, and imaging the subject following administration of the conjugate results in a positive indication of the presence of the targeted cells, this can support the subject has the targeted cells. In such instances, the method can further comprise treating the subject for the disease state associated with the targeted cells.

Uses of the conjugates or compositions hereof in the preparation of a medicament for treating a disease state in a subject are also provided. In certain embodiments, the disease state is cancer. In certain embodiments, the disease state is bladder cancer. In certain embodiments, the disease state is NMIBC.

### EXAMPLES

The present disclosure will be better understood by reference to the following Examples, which are provided as exemplary examples and not by way of limitation.

### Example 1

### Production and Purification of Conjugates for Treatment of Bladder Cancer

The advantages of the conjugates hereof can be exemplified by their application to cancer cells. Given the ready availability of reagents and cell lines, the present disclosure is illustrated in the context of bladder cancer; however, this approach is applicable to any form of cancer.

Invasive bladder cancer is treated by cystectomy-removal of the bladder. Non-muscle invasive bladder cancer (NMIBC) is currently treated with therapeutics of very limited efficacy and poor cancer selectivity. Current anti-bladder cancer drugs do not adapt to tumor cell variability, which narrows the range of patients that can be treated with any degree of success and, thus, results in cancer progression, recurrence, and tumor resistance as well as serious side effects. In addition, a worldwide shortage of Bacillus Calmette Guerin (BCG), which is the adjuvant therapy of choice for NMIBC, underscores the need for other treatment options. Collectively, these factors have resulted in bladder cancer having the highest rate of post-surgery recurrence (≥75%) among malignancies with 25% of cases progressing to invasive bladder cancer resulting in cystectomy. The resulting economic burden on healthcare systems in the U.S. now exceeds $4B/year.

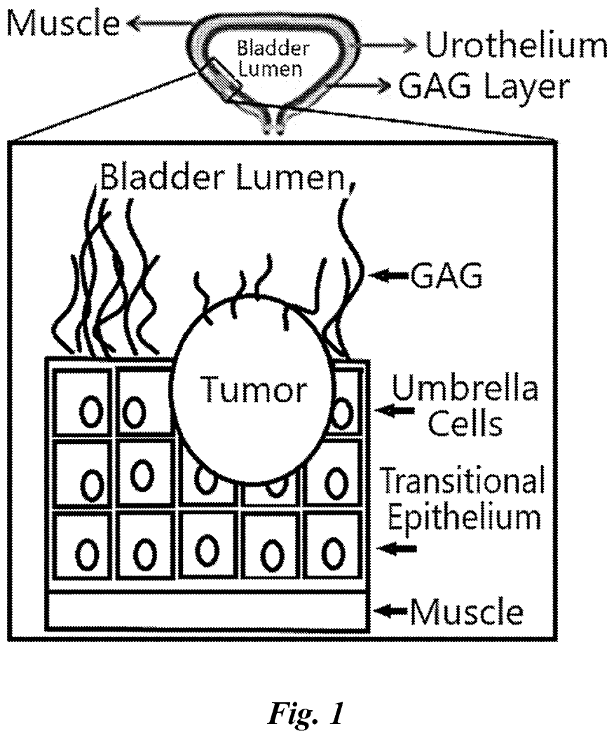

A common practice in NMIBC treatment is the application of therapeutic agents into the lumen of the bladder to take advantage of the differential exposure of tumor cells and normal cells, which are shielded by a glycosaminoglycan (GAG) layer as shown in FIG. 1. Although this treatment approach enables access to tumor cells, diffusion of the therapeutic agents through loose tumor cell junctions enables the anti-cancer therapeutic agents to come into contact with normal (e.g., healthy) cells/tissues as well as blood vessels. Even if the administration of the therapeutic agent is limited and transient, if the tumor regresses, the likelihood that the agent will come into contact with normal cells/tissues increases.

Potential adverse effects to normal cells/tissues can be diminished by utilizing an agent that is selective for cancer cells. This is particularly important when the expression of cell-surface receptors can vary within a tumor, between tumors in a single patient, and between tumors in different patients with the same diagnosis.

In view of the above, provided are MV-MS conjugates, which comprise low-affinity binding motifs for interacting with cell-surface receptors on cancer cells to yield high avidity. In contrast, low densities of such cell-surface receptors on normal cells do not allow avidity-driven binding, causing the MV-MS conjugates to interact with the cell-surface receptors on normal cells with low affinity, i.e., very weakly and transiently, if at all. In other words, the MV-MS conjugates show differential strong, persistent interaction with cancer cells, while displaying low affinity for normal cells.

The binding motifs were selected based on the upregulation of two or more cell-surface receptor targets, which are selected from a group of strategically chosen receptors that can be simultaneously bound by the MV-MS conjugates. As described above, binding of receptor microclusters can induce endocytosis of the conjugate and, consequently, intracellular delivery of the active agent of which the conjugate is comprised. Multivalent binding induces the formation of high local cargo densities, which increases the number of endocytic sites and accelerates initiation and maturation of endocytic vesicles.

MV-MS conjugates were prepared by combining verified low-affinity binding peptides against FGFR3 (Jin et al., FGFR3 signaling and reverses the lethal phenotype of mice mimicking human thanatophoric dysplasia, Human Molecular Genetics 21(26): 5443-55 (2012)), Her2 (Florczak et al., Cellular uptake, intracellular distribution and degradation of Her2-targeting silk nanospheres 2019:6855-6865 (2019)), IL-4Rα (Permpoon et al., Inhibition of tumor growth against chemoresistant cholangiocarcinoma by a proapoptotic peptide targeting interleukin-4 receptor Molecular Pharmaceutics 17(11): 4077-4088 (2020)), and EGFR (Li et al., Identification and characterization of a novel peptide ligand of epidermal growth factor receptor for targeted delivery of therapeutics, FASEB J 19(14): 1933-2088 (2005)) using methodologies commonly known in the art.

Each of the at least four binding motifs comprised VSPPLTLGQLLS (SEQ ID NO: 1) (a binding motif for FGFR3, designated “F”), FCGDGFYACYMDV (SEQ ID NO: 2) (a binding motif for Her2, designated “H”), KLAKLAKKLAKLAK (SEQ ID NO: 3) (a binding motif for IL-4Rα, designated “I”), and YHWYGYTPQNVI (SEQ ID NO: 4) (the binding motif for EGFR, designated “E”). The peptide “E” had a binding affinity for EGFR of less than about 500 M, such as 459 μM. The peptide “H” had a binding affinity for Her2 of less than about 0.5 μM, such as 0.3 μM. The peptide “F” had a binding affinity for FGFR3 of about 1 μM, such as 1 μM. The peptide “I” had a binding affinity for IL-4Rα of less than about 60 μM, such as 55.9 μM.

A linker was positioned between each adjacent binding motif and each linker was approximately 7 nm in length and flexible, and the conjugates further comprised an HA-tag for immunodetection. Each linker had an amino acid sequence independently selected from GRAQGKAQG (SEQ ID NO: 5), GQAKGQARG (SEQ ID NO: 6), GKQAGRQAG (SEQ ID NO: 7), and GQRAGQKAG (SEQ ID NO: 8).

Two conjugates were prepared, purified, and tested, the first comprising SEQ ID NO: 9 (designated “P1: E-I-H-F”), and the second comprising SEQ ID NO: 10 (designated “P2: H-E-F-I”) (FIG. 3C). Peptides “P1” and “P2” covered all receptor micro-clustering possibilities for NMIBC to achieve multiple specificity and address patient and tumor variability (see, e.g., FIG. 3B).

The tested conjugates targeted bladder cancer cells and were internalized. See Coon et al. (2012), supra, for description of methodologies. See, e.g., FIGS. 3D and 3E. Given interspecies receptor conservation, the conjugates targeted cells of human, mouse and canine origin (FIG. 3D), and were internalized in GFP-Rab5-positive endosomes (see, e.g., FIG. 3D, mid-panel set) in a time-dependent manner (FIG. 3E). In contrast to non-specific interactions, preliminary results showed time-dependence for the binding of the ligands to MB49 cells (see, e.g., FIG. 3F).

### Example 2

### Cell-Free Binding Assay

To determine binding of the conjugate comprising a nanoparticle (MV-MS-NP) to bladder cancer cells as compared to control cells, different concentrations of fluorescent nanoparticles decorated or not with the conjugates hereof were incubated with cells from a testing panel at 4° C. (to avoid uptake) for 45 minutes. Jack et al. (2020), supra. After washing, binding was measured by flow cytometry, quantitative microscope and quantitative Western blotting (FIG. 3G). To provide an additional and independent method for binding determination, this assay used isolated membranes from bladder cancer cells; therefore, it lacked the confounding effect of uptake over binding.

To measure rate and extent of MV-MS-NP uptake by bladder cancer cells as compared to control cells, different concentrations of fluorescent nanoparticles decorated or not with the conjugates hereof were incubated with cells from a testing panel at 37° C. for different times (0-30 minutes). After acidic wash to eliminate non-internalized nanoparticles, the internalized fraction was assessed by flow cytometry, quantitative microscopy and quantitative Western blotting.

To determine the extent of MV-MS-NP mediated delivery of cytotoxic load to bladder cancer cells versus control cells, the procedure described above was performed using MV-MS-NPs loaded with or not MMC and a 30 minute fixed exposure time, followed by washes and incubation in complete media. After 48 hours, viability was measured by MTT assays. Jack et al. (2020), supra.

### Example 3

### Receptor Concentration and Receptor Coincidence Detection On Conjugate Binding in Cancer Cells

An ELISA assay was designed to test the ability of the ligands for self-adjusting affinity (FIG. 3H). Purified EGFR and/or Her2 extracellular domain (ECD) produced by bladder cancer cells and engineered to be His₆-tagged and soluble was immobilized on anti-His₆ plates. While normal cells were emulated by low ECD density, cancer cells, which displayed upregulated levels of one or two receptors, were mimicked by high density of one ECD (amount of a single EGFR ECD=amount of a single Her2 ECD) or two ECDs (EGFR ECD+Her2 ECD=amount of either single ECD) (FIG. 3H).

Re-binding caused by high density of one ECD led to higher conjugate retention than normal cell levels, but coincidence detection of two ECDs revealed a substantial avidity effect causing superior binding of MV-MS conjugates. Non-linear regression of these preliminary results produced estimates for observed affinities. Specifically, while for low ECD density, peptides bound with low affinity (>6 μM), the rebinding effect due to high ECD density led to an effective affinity of ˜ 0.1 μM, and coincidence detection revealed high avidity (<0.5 nM).

### Example 4

### MV-MS-Mediated Delivery of Nanoparticles to Bladder Cancer Cells

Nanomolar concentrations of MV-MS conjugates were highly efficient for binding and delivery of nanoparticles to bladder cancer cells (FIG. 3I). The conjugates were stable, and only minimal amounts of agent were required to induce substantial uptake by cancer cells.

Normal (labeled A) and MB49 wild type cancer (labeled B) cells isolated from bladder urothelium were disaggregated and cultured in vitro. Cultured cells were incubated with different concentrations of the P2 MV-MS conjugate and the presence of cell-bound peptide was investigated by immunostaining with an anti-HA antibody using the methodologies described in Example 1.

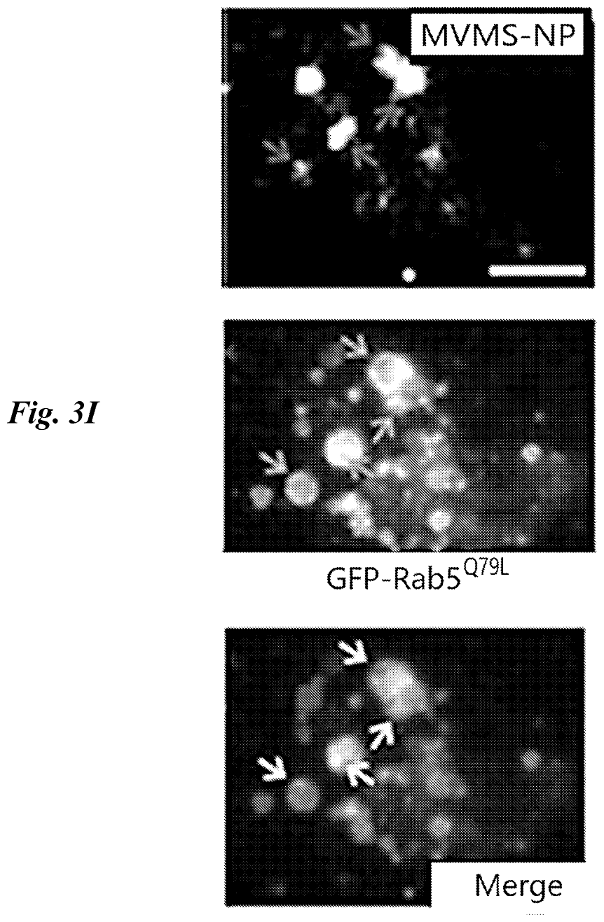

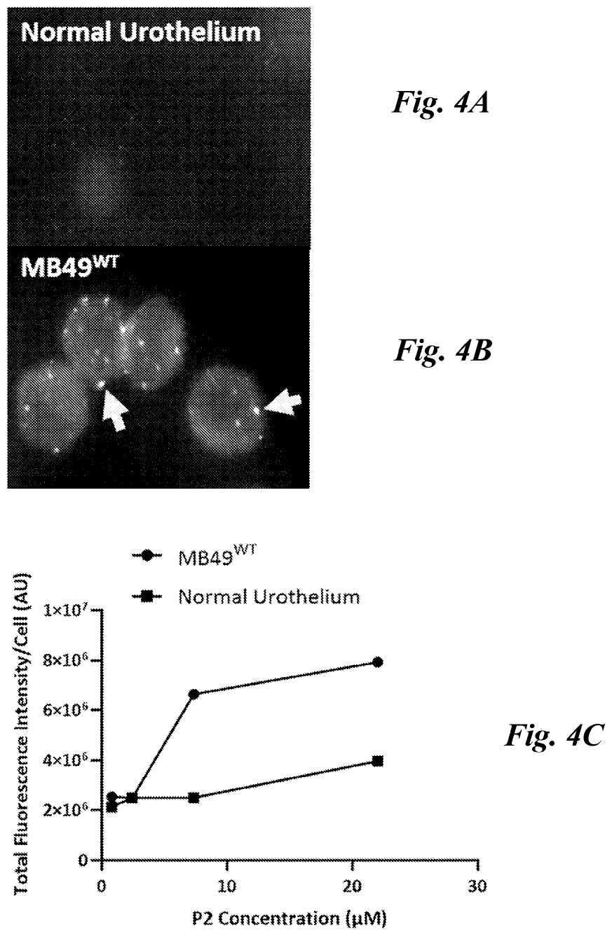

The P2 conjugate demonstrated a marked selectivity for cancer cells with no detectable binding or uptake to normal urothelium (FIGS. 4A-4C).

### Example 5

### In Vitro Binding Studies

The in vitro ability of the conjugates to bind, be internalized by, and deliver a cytotoxic load to different cohorts of cells is assessed. The cohorts include dogs bearing spontaneous bladder tumors, a bladder cancer mouse orthotopic model bearing tumors of engineered expression profiles (i.e., those lacking upregulation of several receptors and/or expressing different combinations of FGFR3, EGFR, Her2, and IL4Rα), and immortalized normal urothelium and unmodified MB49^{LE} cell lines as controls, as well as disaggregated cells taken from resected spontaneous bladder cancer tumors from canine and human subjects with different expression profiles.

To measure rate of uptake of the conjugates by bladder cancer versus control cells, serum-starved cells are incubated for 45 minutes at 4° C. with selected conjugates at 100 μM. Following elimination of unbound conjugates by ice-cold washes, 37° C. warmed media is added, and cells are incubated at 37° C. for different time periods (ranging between 0-30 minutes) to allow uptake. Agent bound but not internalized during this incubation period is stripped off using acidic wash at 4° C.

The conjugate's internalized fraction is assessed by quantitative Western blotting, flow cytometry and quantitative microscopy. For the latter, after elimination of the conjugate not internalized, samples are fixed, and uptake measured by determining cell-associated fluorescence intensity (Image J) inside GFP-Rab5^{Q79L}-labeled endosomes, in samples and controls. Ligand can be detected based on the presence of HA tag at the C-terminus of the conjugate. Cell density is determined by counting DAPI structures≥7 microns in diameter.

A similar procedure is followed for binding of conjugate-decorated nanoparticles (NP). Nano-liposomas (50 nm in diameter to fit in endocytic vesicles) pH-sensitive (PEG-stabilized, to deliver the encapsulated cytotoxic drug into the cytosol from acidic compartments) are selected as NP carriers and prepared pursuant to known protocols.

Briefly, a chloroform solution of DPPC:Cholesterol:DSG-PEG2K-NTA:DSPE-Cy5 (64.46:35:0.04:0.5) in 13 μmol total lipid (this proportion is estimated to yield NP with PEG2K-NTA-His₆-MV-MS complexes separated by 25 nm based on nanocarriers of 50 nm diameter) is prepared. The solution is carefully evaporated under a flow of N₂ gas, to produce an even, thin film. The film is placed under a 50 μm Hg vacuum for 3 hours to remove trace solvent impurities, and thereafter hydrated in 4 ml of 15 mM 4-(2-hydroxyethyl)-piperazineethanesulfonic acid (HEPES) (both with and without 0.4 mg/ml Mitomycin C (MMC); 50% encapsulation efficiency) via 10 freeze-thaw-vortex cycles. The resulting nanocarrier solution is extruded 7 times at 50-55° C. through 3 stacked polycarbonate filters of 800 nm, 200 nm and 50 nm pore diameter using an extruder device charged with 200-300 psi N₂ pressure. The resulting Ni²⁺:trisNTA content in NPs is monitored by Inductively-Coupled Plasma-Mass Spectroscopy (ICP-MS) in each formulation.

The NTA-NPs and His₆-ligands (at a 1:20 ratio) are incubated at room temperature for 1 hour, followed by elimination of unbound peptides. Retention of His₆-ligands is controlled as previously described, which leads to ≤10% loss of ligand from NPs in 24 hours incubation at room temperature. Use of low NTA density (see above) and tags of ≤6 histidines can decrease non-specific binding and increase NP stability.

### Example 6

### In Vivo Binding Studies

The in vivo anti-tumor activity is assessed of the conjugates in dogs bearing spontaneous bladder tumors, a bladder cancer mouse orthotopic model bearing tumors of engineered expression profiles (i.e., those lacking upregulation of several receptors and/or expressing different combinations of FGFR3, EGFR, Her2, and IL4Rα), and immortalized normal urothelium and unmodified MB49^{LE} cell lines as controls. In the mice cohort, tumors were generated by bladder implantation of MB49^{LE} cells stably expressing targeted receptors after electrocautery.

Briefly, using a limited number of animals (n=6 for each test cohort), tumor size in the mice cohort is determined for each animal after treatment with the conjugate or a placebo, and tumor size in the dog cohort is determined for each animal both pre- and post-treatment with the conjugates.

The penetration extent for each agent is also determined by immunohistochemistry in tumor biopsies after treatment.

### General

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

The invention illustratively described herein may be suitably practiced in the absence of any element(s) or limitation(s), which is/are not specifically disclosed herein. Thus, for example, each instance herein of any of the terms “comprising,” “consisting essentially of,” and “consisting of” may be replaced with either of the other two terms. Likewise, the singular forms “a,” “an,” and “the” include plural references unless the context clearly dictates otherwise. Thus, for example, references to “the method” includes one or more methods and/or steps of the type, which are described herein and/or which will become apparent to those ordinarily skilled in the art upon reading the disclosure.

The terms “about” or “approximately” when referring to a number or a numerical value or range (including, for example, whole numbers, fractions, and percentages) are used interchangeably herein and each means that the number or numerical range referred to is an approximation within experimental variability (or within statistical experimental error) and thus the numerical value or range can vary between up to 10% of the stated number or numerical range (e.g., +/−5% to 10% of the recited value) provided that one of ordinary skill in the art would consider equivalent to the recited value (e.g., having the same function or result). The terms “about” and “approximately” can allow for a degree of variability in a value or range, for example, within 90%, within 95%, 99%, 99.5%, 99.9%, 99.99%, or 99.999% or more of a stated value or of a stated limit of a range, inclusive of the specified end points. Each value or range of values preceded by the term “about” is also intended to encompass the embodiment of the state absolute value or range of values.

The terms and expressions, which have been employed, are used as terms of description and not of limitation. In this regard, where certain terms are defined and are otherwise described or discussed elsewhere in the “Detailed Description,” all such definitions, descriptions, and discussions are intended to be attributed to such terms. There also is no intention in the use of such terms and expressions of excluding any equivalents of the features shown and described or portions thereof. Furthermore, while subheadings, may be used in the “Detailed Description,” such use is solely for ease of reference and is not intended to limit any disclosure made in one section to that section only; rather, any disclosure made under one subheading is intended to constitute a disclosure under every other subheading.

It is recognized that various modifications are possible within the scope of the claimed invention. Thus, although the present invention has been specifically disclosed in the context of preferred embodiments and optional features, those skilled in the art may resort to modifications and variations of the concepts disclosed herein. Such modifications and variations are considered to be within the scope of the invention as claimed herein.

1. A conjugate comprising:
(a) at least two or more binding motifs, each of which binds a different cell-surface molecule which is overexpressed or selectively expressed on a targeted cell, wherein adjacent binding motifs are separated from each other by a linker; and
(b) an active agent, which can be endocytosed by a targeted cell to which the conjugate binds;
wherein when the conjugate comprises more than one linker, the linkers can be the same or different from each other; or
a pharmaceutically acceptable salt thereof.

2. The conjugate of claim 1 comprising a structure of Formula I: 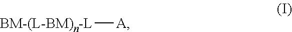
or a pharmaceutically acceptable salt thereof, wherein:
each BM is one of the at least two binding motifs;
each L is a linker;
n is 1-5; and
A is the active agent.

3. (canceled)

4. The conjugate of claim 1, wherein the targeted cell is a a cancerous cell.

5. (canceled)

6. The conjugate of claim 1, wherein the conjugate comprises at least four binding motifs, each of which binds a different cell-surface molecule which is overexpressed or selectively expressed on a targeted cell.

7. The conjugate of claim 6, wherein each cell surface molecule is selected from the group consisting of a transporter, a receptor, a cell surface receptor, and a cell-cell communication protein.

8-9. (canceled)

10. The conjugate of claim 2, wherein:
the conjugate comprises at least four binding motifs, each of which binds a different cell-surface receptor which is overexpressed or selectively expressed on a targeted cell and is selected from the group consisting of fibroblast growth factor receptor 3 (FGFR3), Her2, interleukin-4 receptor alpha (IL-4Rα), and epidermal growth factor receptor (EGFR); and
the targeted cell is a cancerous cell.

11. The conjugate of claim 1, wherein the binding motif for FGFR3 is SEQ ID NO: 1 or a functional variant thereof (designated “F”), the binding motif for Her2 is SEQ ID NO: 2 or a functional variant thereof (designated “H”), the binding motif for IL-4Rα is SEQ ID NO: 3 or a functional variant thereof (designated “I”), and the binding motif for EGFR is SEQ ID NO: 4 or a functional variant thereof (designated “E”).

12. The conjugate of claim 1, wherein each linker is (a) approximately 5 nm to 15 nm in length, (b) approximately 7-10 nm in length, or (b) approximately 7 nm in length and flexible.

13-14. (canceled)

15. The conjugate of claim 1, wherein each linker has an amino acid sequence independently selected from SEQ ID NO: 5, SEQ ID NO: 6, SEQ ID NO: 7, and SEQ ID NO: 8.

16. The conjugate of claim 15, wherein each linker is approximately 7 nm in length and flexible.

17. The conjugate of claim 1, wherein the active agent is an anti-cancer therapeutic agent or an imaging agent and/or the active agent is attached to a nanoparticle or encapsulated in a liposome, wherein the nanoparticle or liposome is attached to a linker of the conjugate.

18-19. (canceled)

20. The conjugate of claim 1, comprising SEQ ID NO: 9 or a functional variant thereof, or SEQ ID NO: 10 or a functional variant thereof.

21. (canceled)

22. The conjugate of any claim 1, wherein each binding motif is a low-affinity binding motif.

23. A composition comprising:
(a) a conjugate comprising:
at least two or more binding motifs, each of which binds a different cell-surface molecule which is overexpressed or selectively expressed on a targeted cell, wherein adjacent binding motifs are separated from each other by a linker; and
an active agent, which can be endocytosed by a targeted cell to which the conjugate binds;
wherein when the conjugate comprises more than one linker, the linkers can be the same or different from each other; or
a pharmaceutically acceptable salt thereof; and
(b) a pharmaceutically acceptable carrier.

24. The composition of claim 23, which comprises two conjugates, both of which comprise binding motifs that bind the same four cell-surface molecules overexpressed or selectively expressed on a targeted cell but wherein the order of the binding motifs differs between the two conjugates.

25. The composition of claim 24, which comprises (i) a first conjugate comprising SEQ ID NO: 9 or a functional variant thereof and (ii) a second conjugate comprising SEQ ID NO: 10 or a functional variant thereof.

26. A method of selectively targeting a cancerous cell in a subject for endocytosis of an active agent comprising administering to the subject an effective amount of:
(a) a conjugate comprising:
at least two or more binding motifs, each of which binds a different cell-surface molecule which is overexpressed or selectively expressed on a targeted cell, wherein adjacent binding motifs are separated from each other by a linker; and
an active agent, which can be endocytosed by a targeted cell to which the conjugate binds;
wherein when the conjugate comprises more than one linker, the linkers can be the same or different from each other; or
a pharmaceutically acceptable salt thereof, or
(b) a composition comprising (i) the conjugate or a pharmaceutically acceptable salt thereof, and
(ii) a pharmaceutically acceptable carrier;
wherein the active agent of the conjugate or pharmaceutically acceptable salt thereof comprises an anti-cancer agent.

27. (canceled)

28. The method of claim 26, wherein the subject has bladder cancer.

29. (canceled)

30. The method of claim 26 further comprising
imaging the subject.

31. The method of claim 30, wherein imaging the subject comprises radio-imaging, positron emission tomography (PET) imaging, single-photon emission computer tomography (SPECT) imaging, or magnetic resonance imaging and/or the imaging agent comprises:
a metal or isotope suitable for radio-imaging, PET imaging, SPECT imaging, or magnetic resonance imaging; or
a fluorescent imaging agent, a photodynamic imaging agent, or an optical imaging agent.

32-37. (canceled)
